(19) United States
(12) Patent Application Publication
(10) Pub. No.: US 2025/0160337 A1
(43) Pub. Date: May 22, 2025

(54) METHOD OF USING/APPLYING A KERATIN HYDROLYSIS PEPTIDE SOLUTION TO INCREASE PRODUCTION YIELD OF LETTUCE

(71) Applicant: CH Biotech R&D Co., Ltd., Nantou City, Nantou County (TW)

(72) Inventors: Kai XIA, Nantou (TW); Meng-Ying LI, Nantou (TW); Ming-Ru LIOU, Taichung (TW)

(73) Assignee: CH Biotech R&D Co., Ltd., Nantou City, Nantou County (TW)

(21) Appl. No.: 18/645,293

(22) Filed: Apr. 24, 2024

(30) Foreign Application Priority Data
Nov. 17, 2023 (TW) 112144616

(51) Int. Cl. A01N 63/50; A01P 21/00; C12P 21/06

(52) U.S. Cl. CPC A01N 63/50; A01P 21/00; C12P 21/06

(57) ABSTRACT

Present invention teaches the method of using a keratin hydrolysis peptide (“KHP”) solution to enhance lettuce plant's growth and production yield. By selectively choosing specific weights of feathers and water, and treating the mixture to a high-temperature high-pressure hydrolysis process, the resulting solution is confirmed to contain at least 253 peptides and then infused to the soil in which the lettuces are planted; the solution can also be sprayed to the leaf surface of lettuce plants. Optionally, the KHP solution can be diluted by water, as disclosed in the specification, for applying to the soil around the lettuce plants and for spraying to leaf surface of lettuce plants.

### PRIORITY CLAIM TO FOREIGN APPLICATION

Applicant hereby makes priority claim to a Taiwan application, number 112144616, having the Taiwan filing date of Nov. 17, 2023.

### SEQUENCE LISTING/INCORPORATION BY REFERENCE

Table I (in Sequence Listing XML format) shows the at least 253 peptides and its annotated sequences for the solution generated in accordance with the disclosure of this application. The Sequence Listing XML file complies with the WIPO ST.26 requirements. Said XML copy, created on Mar. 17, 2024, is named Table-I-253_sequence and is 216 bytes in size.

Applicant hereby incorporates by reference said Sequence Listing XML file in its entirety as part of the disclosure and specification of the present application.

### BACKGROUND OF THE INVENTION

Present invention disclosed and claimed the method and application of a keratin hydrolysis peptide (“KHP”) solution for increasing the production yield of lettuce, by using the thermally hydrolyzed feather keratin peptide solution to the soil where the young lettuce plants were transplanted in, the above-ground biomass and root health of the lettuce plants are improved, resulting in better photosynthetic capacity and metabolic activity, so as to increase the production yield of lettuce.

The solution, as made per the disclosure herein, can also be sprayed, at the proper dilution ratio, to the lettuce plant's leave's to help with the increase of production yield.

The KHP solution is made by a hydrolysis process using feathers and water, via a high-temperature and high-pressure process, resulting in a solution that has many beneficial applications in the fields of horticulture, agriculture and potentially other farming businesses.

Hydrolyzed keratin has long been used to strengthen hairs, reduce hair splitting and breakage. Other beneficial uses include skin moisturization and wound healing. Keratin hydrolysate has also been known to function as a biofertilizer, boosting plants' growth by enhancing the plants' ability to receive and utilize nutrients, including commonly applied fertilizers.

Lettuce (scientific name: Lactuca Sativa) is a member of the Lactuca genus and the Asteraceae family. It is an annual herbal plant originated around the Mediterranean regions, and spreading to temperate and subtropical areas.

Based upon the Food and Public Health Agency of Taiwan's Executive Yuan, the makeup of lettuce, on a per 100-gram portion, consists primarily of 95% water, 11 Calories, 0.6 g fat, 0.6 g carbohydrate, 0.8 g fiber, 3300 I.U. vitamin A, 0.08 mg vitamin Bi, 20.11 mg vitamin B, 0.01 mg vitamin B6, 15 mg vitamin C, 12 mg sodium, 220 mg potassium, 34 mg calcium, 30 mg phosphorus, 1.2 mg iron, 21 mg magnesium, and 0.3 mg zinc.

The lettuce further contains vitamin E, beta carotin, apple acid, digestive enzymes and trace amount of salt and vitamin K (one of the clotting factors). The beta carotin content is higher than many other green vegetables and is known to have anti-oxidating effect to help with the prevention of cancer cells. Every 100 g of lettuce contains 867.7 mg of beta carotin, higher than contained in cauliflower, bok choy, mustard and other leafy vegetables. The nutritional values of lettuce have been widely recognized.

According to The Food and Agriculture Organization Corporate Statistical Database, the global lettuce harvesting area is about 1.21 million hectors, having total 27 million tons in 2021. The transaction dollar amount in 2021 reached $3.31 million US dollars. Consequently, it has always been a goal to promote the growth of lettuce and to increase the production yield, to boost up the lettuce's economic values.

The present invention's KHP solution and the method of using same is a cost-effective way to improve the growth of lettuce plants and to increase the ultimate production yield. The inventors of present application conducted field tests and confirmed the effectiveness of applying the KHP solution as disclosed and claimed herein.

### SUMMARY OF THE INVENTION

The keratin solution is primarily based upon feather, which contains 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by the mixture of a-amylase and protease to hydrolyze feather waste, resulting in a mixture of amino acids, fatty acids, and sugars. Nurdiawati experimented and adopted certain specific high-temperature and high-pressure setting in the hydrolysis process and discovered that the resulting solution, when mixed with some potassium and other minerals, can boost the growth of Pogostemon cablin and Vigna radiata, as reported in International Journal of Recycling or Organic Waste in Agriculture (8:221-232, 2019).

The inventors of present application, under the aegis of CH Biotech, developed and selected different feather and water compositions to perform the hydrolysis at higher temperature and higher pressure settings, resulting with different keratin hydrolysis peptide (“KHP”) solution that can be used on different crops/plants.

The selected embodiment of present invention uses a mixture of water and feathers, and subject the mixture to a thermal hydrolysis process to create KHP solutions based upon temperature/pressure parameters as noted below.

The inventors used Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The solutions are infused to the soil containing the lettuce plants. Alternatively, the solutions can be sprayed to the leaf surface of the lettuce plants.

The solution can be diluted by water, at 50 to 1000 ratio by volume, and then applied to the soil containing the lettuce plants, and sprayed to the lettuce plant's leaves.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

FIGS. 1A and 1B show the comparisons of above-ground bio-mass weight (1A) and dry weight (1B) of lettuce plants in three groups: Check group (first bar in the group); KHP-1 50× (dotted bar); KHP-1 250× (black bar), at 7 and 14 days after the infusion of the solutions to the soil.

FIGS. 2A and 2B show the comparisons of above-ground bio-mass weight (2A) and dry weight (2b) of lettuce plants in three groups: Check group (first bar in the group): KHP-1 500× (dotted bar); KHP-2 500× (black bar), at 7 and 14 days after leaf spraying and at harvest time.

FIG. 3A shows the comparison of leaf count in three groups: Check group (first bar in the group); KHP-1 50× (dotted bar); KHP-1 250× (black bar), at 7 and 14 days after infusion of the solutions to the soil.

FIG. 3B shows the comparison of leaf surface area in three groups: Check group (first bar in the group); KHP-1 50× (dotted bar); KHP-1 250× (black bar), at 7 and 14 days after infusion of the solutions to the soil.

FIG. 4A shows the comparison of leaf count in three groups: Check group (first bar in the group); KHP-1 500× (dotted bar); KHP-2 500× (black bar), at 7 and 14 days after spraying to the lettuce plant's leaves.

FIG. 4B shows the comparison of leaf surface area in three groups: Check group (first bar in the group); KHP-1 500× (dotted bar); KHP-2 500× (black bar), at 7 and 14 days after spraying to the lettuce plant's leaves.

FIG. 5A shows the comparison of electron transmission rate (ETR) in three groups: Check group (first bar in the group); KHP-1 50× (dotted bar); KHP-1 250× (black bar), at 3 and 7 days after infusion to the soil containing the lettuce plants.

FIG. 5B shows the comparison of chlorophyll counts (SPAD) in three groups: Check group (first bar in the group); KHP-1 50× (dotted bar); KHP-1 250× (black bar), at 3 and 7 days after infusion to the soil.

FIG. 6 shows the comparison of photosynthesis effectiveness by measuring the electron transport rate in three groups: Check group (first bar in the group); KHP-1 500× (dotted bar); KHP-2 500× (black bar), at 7 days after spraying to lettuce leaves.

FIG. 7 shows the comparison of leaf metabolic activeness of three groups: Check group (first bar in the group); KHP-1 50× (dotted bar); KHP-1 250× (black bar), at 7 and 14 days after infusing the solutions to the soil.

FIG. 8 shows the comparison of leaf metabolic activeness of three groups: Check group (first bar in the group); KHP-1 50× (dotted bar); KHP-1 250× (black bar), at 7 and 14 days after spraying to the lettuce plant's leaves.

FIGS. 9A and 9B show the comparisons of root development (9A) and underground bio-mass weight (9B) of three groups: Check group (first bar in the group); KHP-1 50× (dotted bar); KHP-1 250× (black bar), at 7 and 14 days after infusing the solutions to the soil.

FIGS. 10A and 10B show the comparisons of root development (10A) and underground bio-mass weight (10B) of three groups: Check group (first bar in the group); KHP-1 500× (dotted bar); KHP-2 500× (black bar), at 7 and 14 days after spraying the solutions to the lettuce leaf surface, and at time of harvesting.

FIG. 11A shows the comparison of plant height of actual field plants consistent with normal farming practice among five groups: Check group (first bar in the group); KHP-1 250× (dotted bar); KHP-1 500× (black bar); KHP-2 250× (slanting strips); and KHP-2 500× (white bar), at 7, 14, 21, and 28 days after spraying the solutions to the lettuce leaf surface.

FIG. 11B shows the comparison of above-ground weight of actual field plants consistent with normal farming practice among five groups: Check group (first bar in the group); KHP-1 250× (dotted bar); KHP-1 500× (black bar); KHP-2 250× (slanting strips); and KHP-2 500× (white bar), at 7, 14, 21, and 28 days after spraying the solutions to the lettuce leaf surface.

FIGS. 12A and 12B show the comparisons of above-ground fresh weight (12A) and dry weight (12B) on actual field plants consistent with normal farming practice among three groups: Check group (first bar in the group); KHP-1 250× (dotted bar); KHP-1 500× (black bar); KHP-2 250× (slanting strips); and KHP-2 500× (white bar), 9 days after transplantation and 8 days before harvesting when the leaf spraying of the solutions are done.

FIG. 12C show the comparison of harvested box production in an area of 0.00085 hector on actual field plants consistent with normal farming practice among three groups: Check group (first bar in the group); KHP-1 250× (dotted bar); KHP-1 500× (black bar); KHP-2 250× (slanting strips); and KHP-2 500× (white bar), 9 days after transplantation and 8 days before harvesting when the leaf spraying of the solutions are done.

### DETAILED DESCRIPTION OF THE INVENTION

The keratin hydrolysis peptide (“KHP”) solution of present invention is made by a high-temperature and high-pressure process to treat a mixture of water and feathers as shown in the parameters herein.

The mixture ratio, temperature, pressure and duration parameters are shown herein:

| | | | Water content | | | | | |
|---|---|---|---|---|---|---|---|---|
| | Feather | Water | in feather | Pressure | Temp. | Time | Mass | Concen. |
| | (kg) | (kg) | (%) | (kg/cm²) | (° C.) | (min) | (Da) | (ppm) |
| KHP-1 | 66 | 44 | 50% | 16 | 195 | 40 | 593.3~3828.0 | 200000 |
| KHP-2 | 50 | 40 | 50% | 12 | 185 | 80 | 593.3~3508.9 | 301500 |

The hydrolysis process, in the first embodiment (KHP-1) takes the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵ 4.5×10⁵ ppm.

The hydrolysis process, in the second embodiment (KHP-2) takes the steps of:
a. Preparing the KHP solution by mixing 50 kg of feathers whose content is 50% water with 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵ 4.5×10⁵ ppm.

The confirmation of some of the 253 peptides is further done by referencing the BIOPEP-UWM database.

The solution, either the KHP-1 solution or the KHP-2 solution, can then be applied to the soil containing the lettuce plants, as well as spraying to the leaf surface of the lettuce plants. The applied solution can be diluted with water by volume at ratio of 1:50-1,000.

The method of using a keratin hydrolysis peptide (KHP) solution stated above further causes the solution to be diluted with water by volume at the ratio of 1:50-300 for infusing to the soil containing the lettuce plants.

The method of using a keratin hydrolysis peptide (KHP) solution stated above further caused the solution to be diluted with water by volume at the ratio of 1:300-500 for spraying to the lettuce plants' leaves.

The inventors conducted different tests, in controlled rooms and in the field, by defining check group (noted as CHK) and additional test groups that are administered with either KHP-1 or KHP-2, at desired dilution ratios as taught and claimed, at the time/day as noted herein.

The manner of administration of KHP solutions is either by infusing into the soil where the transplanted young letter plants are grown, or by spraying to the lettuce plaints' leaves, at the dilution ratios as disclosed.

Generally, for soil infusion, the dilution ratios are between 50-1,000, with 50-300 being the preferred range. For leaf spraying, the dilution rations are between 100-1,000, with 300-500 being the preferred range.

The specific lettuce plant chosen for the tests done by the inventors is the Green Romaine lettuce. The seeds are first planted into a starting pot and placed in a greenhouse for 14 days, with day/night temperatures set at 17° C./15° C. Then the plant is transplanted to a 7-inch growing pot and the application of the KHP solutions start 7 days after the transplantation, either by soil infusion or by leaf spraying. The application is done weekly, for 3 consecutive times. Tests were done at different times as noted, including at harvesting time.

The inventors use both embodiments, with different dilution ratios as noted: KHP-1 50× denotes using KHP-1 solution with water dilution of 50 times. KHP-1 100× denotes using KHP-1 solution with water dilution of 100 times. KHP-1 500× denotes using KHP-1 solution with water dilution of 500 times. KHP-2 500× denotes using KHP-2 solution with water dilution of 500 times. Other dilution ratios may be used as additionally noted.

For comparison, a check group (noted as CHK) of lettuce plants are planted under normal condition with water being given, without any of the KHP solution.

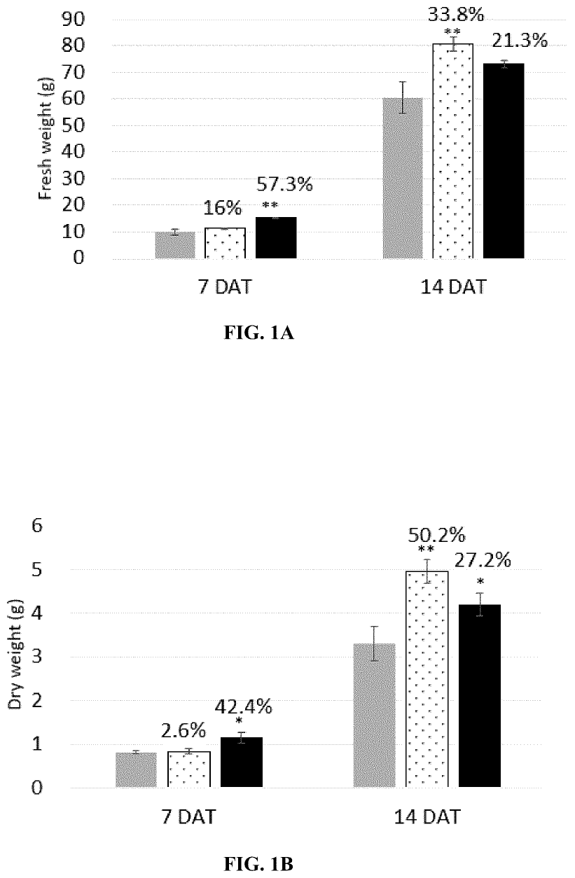

The inventors did experiment with soil infusion of KHP-1 50× and KHP-2 250× solutions, and take measurements 7 and 14 days after infusion. The above-ground bio mass weight increases are reflected in FIGS. 1A and 1B.

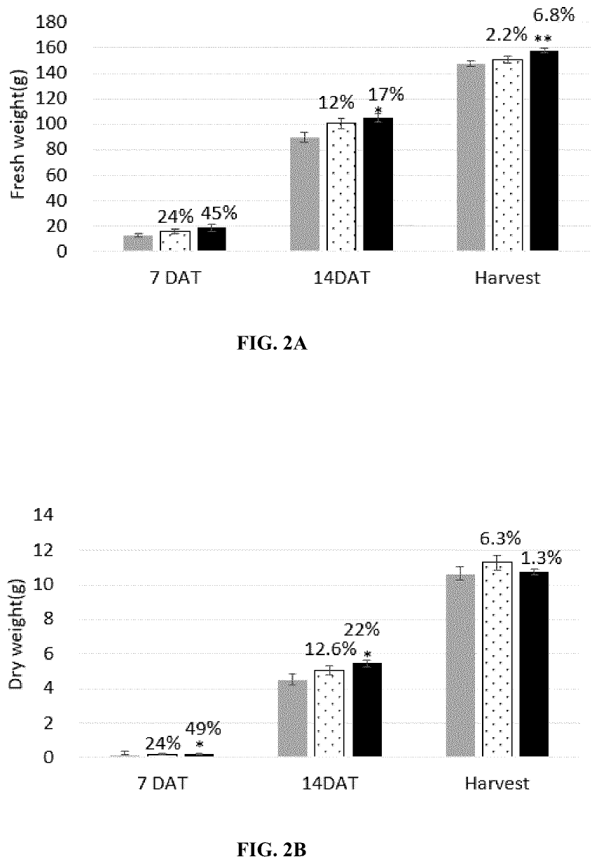

The application of the KHP-1 500× and KHP-2 500× solutions by leaf spraying also substantially boosted the above-group bio mass of the lettuce plants. Relative to the Check group where only water is given, the 7 and 14 day measurements, and the time of harvest measure, are noted in FIGS. 2A and 2B. The fresh weight increases show 24%, 12% and 2% for the KHP-1 500× group, and 45%, 17% and 6.8% for the KHP-2 500× group, as shown in FIG. 2A. The dry weight increases show 24%, 12.6% and 6.3% for the KHP-1 500 group, and 49%, 22% and 1.3% for the KHP-2 500 group.

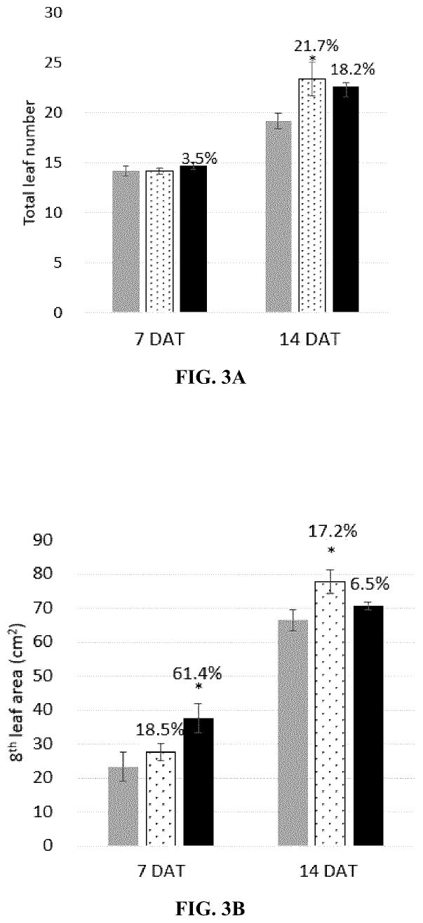

Among the test items, the comparison on the leaf count and leaf surface area would be critical to the goal of increasing production due to the direct connection with the effectiveness of photosynthesis performed by the lettuce plants. The leaf count measured at the 7 and 14 days after infusion of the KHP-1 solution show that, per the analysis by the WinFOLIA Pro LA 2400 system, the KHP-1 50× group has 21.7% more leaf count than the CHK group; the KHP-1 250× group has 18.2% more leaf than the CHK group. This fact is reflected in FIG. 3A.

The leaf area's increase is also pretty obvious after the soil infusion. The KHP-1 50× group has leaf area increase of 18.5% 7 days after infusion and 17.2% increase 14 days after infusion. The KHP-1 250× group has leaf area increase of 61.4% 7 days after infusion and 6.5% increase 14 days after infusion. This fact is reflected in FIG. 3B.

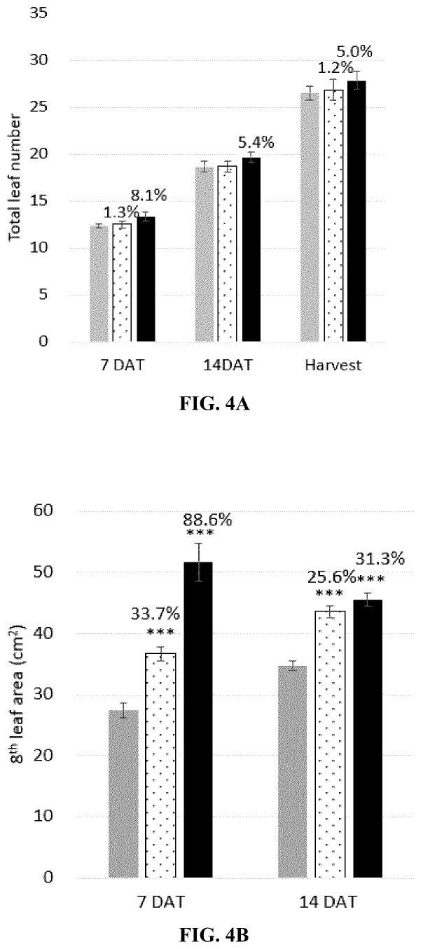

The leaf count measured 7 days, 14 days after leaf spraying of KHP-2 500× solution, and at time of harvesting, also show increase of 8.1%, 5.4% and 5%. The leaf count measured 7 days, 14 days after leaf spraying of KHP-1 500× solution show increase of 33.7% and 25.6%. When the KHP-2 500× is used to do the leaf spraying, the leaf count measured should increase of 88.6% and 31.3%. These facts are reflected in FIGS. 4A and 4B.

Among the test items, the results and comparisons about photosynthesis activities of the lettuce plants. The inventors used a Junior PAM (Walz GmbH) to measure the electron transport rate. A SPAD 502 Plus Chlorophyll Metter (model number 2900, Spectrum Technologies) is used for SPAD value analysis, which positively correlates to chlorophyll counts and the activities of photosynthesis.

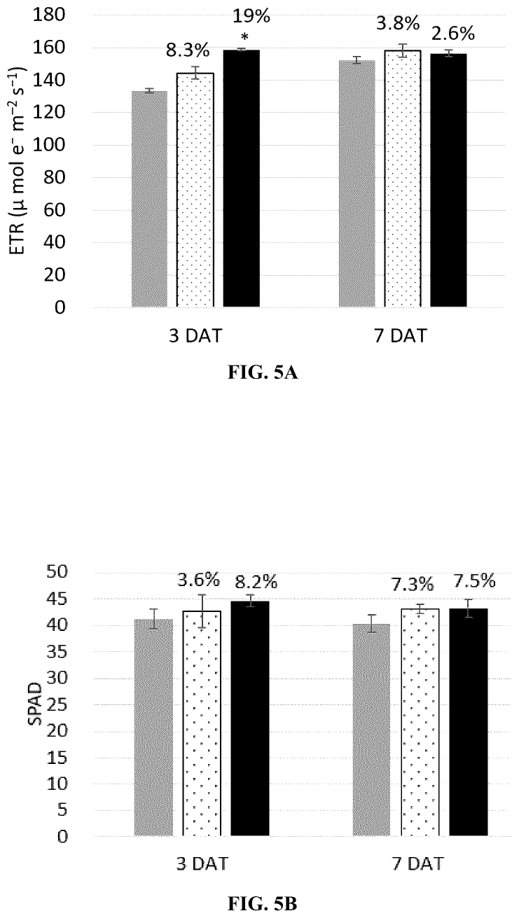

FIGS. 5A and 5B shows the comparison of electron transmission rate (ETR) and the SPAD count in three groups: Check group (first bar in the group); KHP-1 50× (dotted bar); KHP-1 250× (black bar), at 3 and 7 days after infusion to the soil containing the lettuce plants. The increases are noted as 8.3% and 3.8%, and the SPAD count increased 3.6% and 7.3% in the KHP-1 50× group. The increases are noted as 19% and 2.6%, and the SPAD count increased 8.2% and 7.5% in the KHP-1 250× group.

The measurement of ETR represents the speed of electron being transmitted in the photosynthesis link, a direct indication of the photosynthesis efficiency, while the SPAD count indicates the number of chlorophyll, having a positive correlation to the photosynthesis activities. As shown in the tests and figures above, the KHP solutions, whether applied by soil infusion or by leaf spraying, substantially boosted the ETR and SPAD count, leading to higher photosynthesis activities of the lettuce plants.

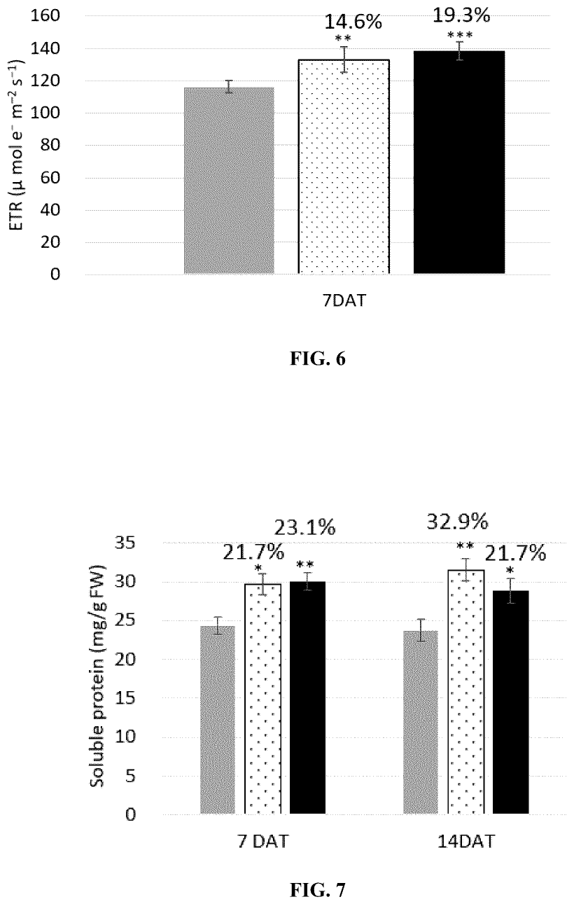

When using leaf spraying, as shown in FIG. 6, the KHP-1 500× group show ETR increase of 14.6% and KHP-2 500× group shows ETR increase of 19.3%.

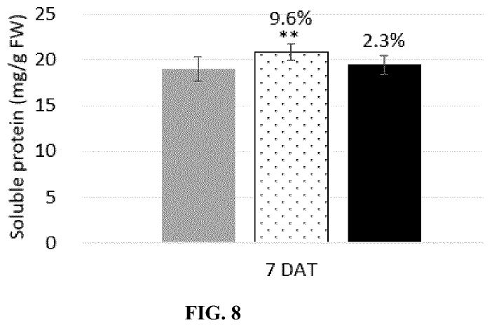

The inventors also test the metabolic activities of the lettuce plants by measuring soluble protein, which is an important biophysical index tied to a plant's metabolism. The inventors took measurement on the 7^{th} and 14^{th} days after soil infusion, and another measurement on the 7^{th} day after leaf spraying, pick the 6^{th} leaf to conduct protein extraction and obtained the soluble protein count using the Spark multimode reader (Tecan). As reflected in FIG. 7, the soluble protein increased 21.7% and 32.9% on the 7^{th} and 14 days after infusion when administered with KHP-1 50× solution; the increase became 23.1% and 21.7% when administered with KHP-1 250× solution.

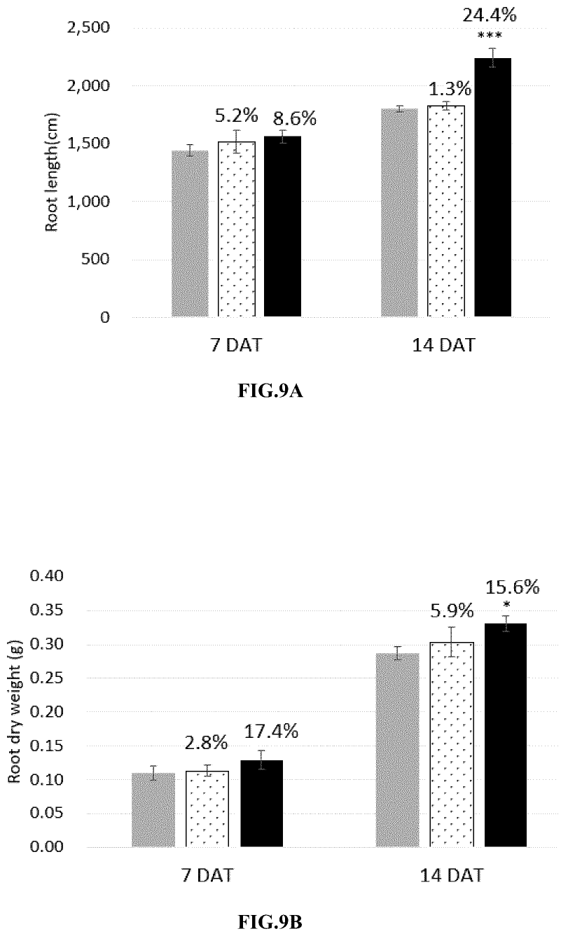

When the administration is by leaf spraying, the soluble protein increased 9.6% with KHP-1 500× solution, and increased 2.3% with KHP-2 500× solution, as reflected in FIG. 8.

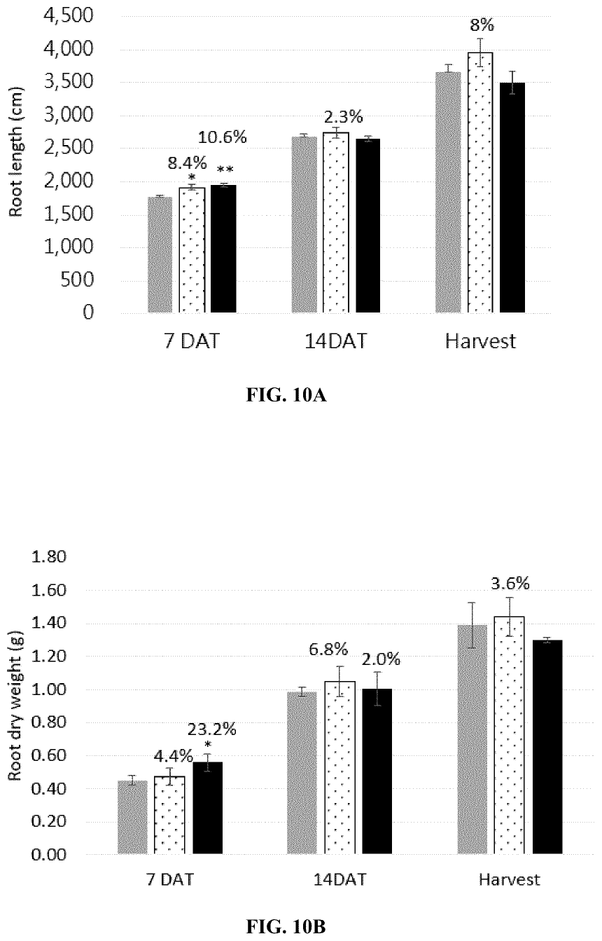

The inventors conducted tests to measure the KHP solution effects on underground bio-mass and root development. The test result showed that soil infusion of KHP-1 250× solution caused the root length to increase 24.4%, when measured at 14^{th} day after soil infusion, as reflected in FIG. 9A.

The infusion of KHP-1 250× solution caused the underground bio-mass weight to increase, at 7 and 14 days after infusion, to increase 17.4% and 15.6%, as reflected in FIG. 9B.

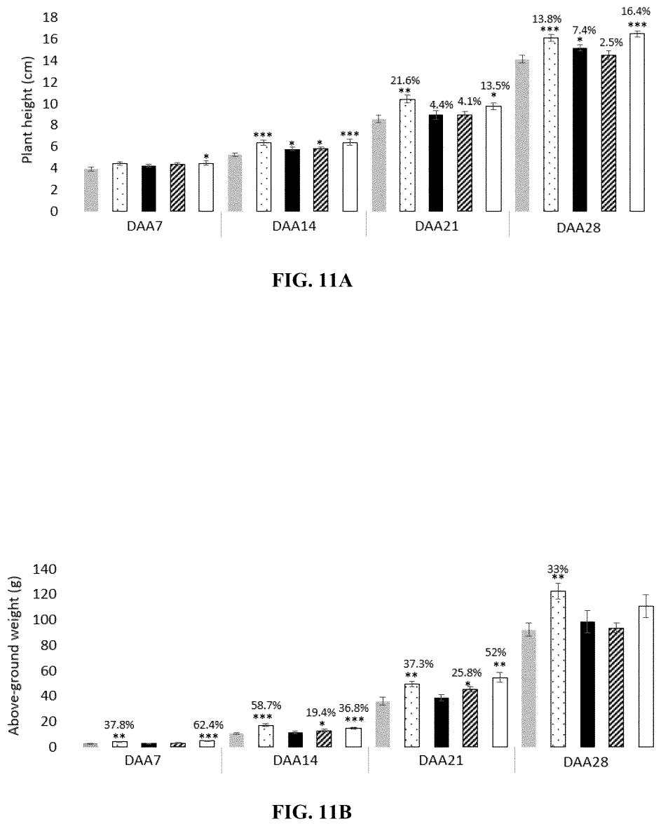

When KHP-1 500× and KHP-2 500× solutions were leaf sprayed on the 7^{th} day, the root length increases are 8.4% and 10.6%, as noted in FIG. 10A.

When KHP-1 500× and KHP-2 500× solutions were leaf sprayed on the 7^{th} day, the underground bio-mass weight increased 4.4% and 23.2%. This is reflected in FIG. 10B.

As shown above, both soil infusion and leaf spraying of the KHP solutions help with the root growth and underground bio-mass development, boosting up the lettuce plant's ability for nutrient intake.

In addition to the tests done in a controlled environment (greenhouse), the inventors also conducted field tests under conditions experienced by normal farming operations. The field test sites are set around YunLin County, selecting two 5 meter by 1.7 meter rectangular patches that are not adjacent to each other for planting the lettuce plants.

The species of “FuShan” lettuce is chosen, for leaf spraying by KHP-1 250× and 500× and KHP-2 250× and 500× solutions on the 9^{th} day after transplantation and 8^{th} day before harvesting, with a check (CHK) group receiving water only. The lettuce plants are harvested after 45 days for taking some height and weight measurements.

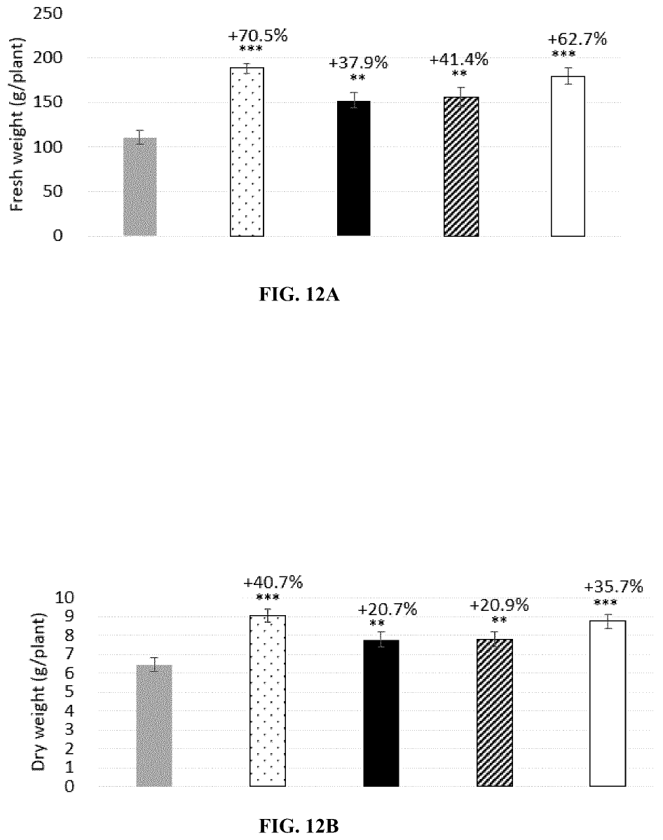

As shown in FIG. 11A, the height of the in-field lettuce plants increased substantially, when measured on at 7, 14, 21, and 28 days after spraying the solutions to the leaf surface. The height increase is between 2.5%-16.4% for all KHP groups on the 28^{th} day relative to the CHK group. FIG. 11B shows the above-ground bio-mass weight increased, measured on the 28^{th} day, to a substantial 33% when applied with KHP-1 250× solution.

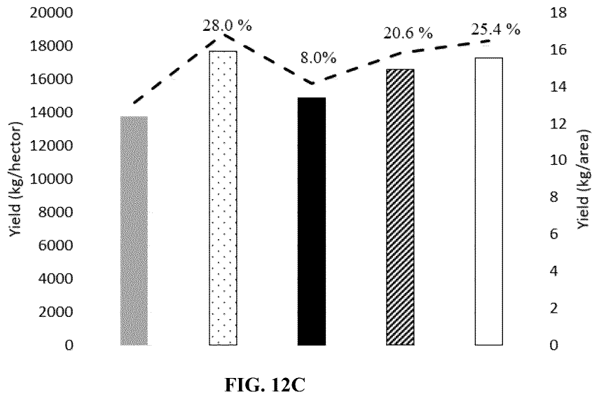

In addition to height comparison, the weight of the harvested leaves, measured by a “per plant” basis, is a simple and direction correlation to the production yield. As shown in FIGS. 12A and 12B, the application of leaf spraying with the solutions of KHP-1 250×, KHP-1 500×, KHP-2 250×, and KHP-2 500× all show substantial boost to the above-ground growth of the lettuce plants. Compared to the CHK group, the fresh weight increase is noted as 70%, 37.9%, 41.4% and 62.7% in FIG. 12A; the dry weight increase is noted as 40.7%, 20.7%, 20.9% and 35.7% in FIG. 12B.

When the production yield increase computed on a weight-per-unit-area basis, the increases relative to the CHK group, respectively for the 4 groups of KHP-1 250×, KHP-1 500×, KHP-2 250×, and KHP-2 500×, are 28%, 8%, 20.6% and 25.4% as shown in FIG. 12C.

As has been proven by the field tests, and the scientific analysis/measurement done by the inventors, the method of creating the KHP solution and the method of application to will help with the growth and the production yield of lettuce plants when the photosynthetic capacity and metabolic activities are greatly boosted by the teachings as disclosed herein.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method of using a keratin hydrolysis peptide (KHP) solution to increase the production yield of lettuce, comprising the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to lettuce plants where the solution is diluted with water by volume ratio of 1:50-1,000.

2. The method of using a keratin hydrolysis peptide solution of claim 1 wherein the application is by infusing to the soil containing the lettuce plants the solution diluted with water by volume at the ratio of 1:50-300.

3. The method of using a keratin hydrolysis peptide (KHP) solution of claim 1 wherein the application is by spraying to the lettuce leaves the solution diluted with water by volume at the ratio of 1:300-500.

4. A method of using a keratin hydrolysis peptide (KHP) solution to enhance the production yield of lettuce, comprising the steps of:
a. Preparing the KHP solution by mixing 50 kg of feathers whose content is 50% water and 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to lettuce plants where the solution is diluted with water by volume ratio of 1:50-1,000.

5. The method of using a keratin hydrolysis peptide solution of claim 4 wherein the application is by infusing to the soil containing the lettuce plants the solution diluted with water by volume at the ratio of 1:50-300.

6. The method of using a keratin hydrolysis peptide (KHP) solution of claim 4 wherein the application is by spraying to the lettuce leaves the solution diluted with water by volume at the ratio of 1:300-500.


SEQUENCE LISTING (253 sequences)

SEQ ID NO: 1 - protein, 6 aa, Gallus gallus
FGLSGL

SEQ ID NO: 2 - protein, 7 aa, Gallus gallus
AAVGSSL

SEQ ID NO: 3 - protein, 7 aa, Gallus gallus
TAVGSST

SEQ ID NO: 4 - protein, 6 aa, Gallus gallus
LSLSSQ

SEQ ID NO: 5 - protein, 7 aa, Gallus gallus
FGISGLG

SEQ ID NO: 6 - protein, 6 aa, Gallus gallus
PILSSF

SEQ ID NO: 7 - protein, 7 aa, Gallus gallus
SLLSQGA

SEQ ID NO: 8 - protein, 6 aa, Gallus gallus
FNLRGA

SEQ ID NO: 9 - protein, 8 aa, Gallus gallus
SAAVGSSL

SEQ ID NO: 10 - protein, 6 aa, Gallus gallus
SRVVIQ

SEQ ID NO: 11 - protein, 6 aa, Gallus gallus
SRVVLE

SEQ ID NO: 12 - protein, 8 aa, Gallus gallus
GFGISGLG

SEQ ID NO: 13 - protein, 6 aa, Gallus gallus
SRFSGR

SEQ ID NO: 14 - protein, 6 aa, Gallus gallus
RVVIQP

SEQ ID NO: 15 - protein, 8 aa, Gallus gallus
SAAVGSIL

SEQ ID NO: 16 - protein, 7 aa, Gallus gallus
IVVIGHV

SEQ ID NO: 17 - protein, 9 aa, Gallus gallus
TAVGSSTSA

SEQ ID NO: 18 - protein, 7 aa, Gallus gallus
RVVIQPS

SEQ ID NO: 19 - protein, 7 aa, Gallus gallus
SRVVIQP

SEQ ID NO: 20 - protein, 7 aa, Gallus gallus
PQGEQLQ

SEQ ID NO: 21 - protein, 9 aa, Gallus gallus
RAGAGPPSP

SEQ ID NO: 22 - protein, 7 aa, Gallus gallus
DSRVVIQ

SEQ ID NO: 23 - protein, 8 aa, Gallus gallus
PGPILSSF

SEQ ID NO: 24 - protein, 9 aa, Gallus gallus
PISSGGFGI

SEQ ID NO: 25 - protein, 8 aa, Gallus gallus
ISGLGSRF

SEQ ID NO: 26 - protein, 10 aa, Gallus gallus
SGGFGISGLG

SEQ ID NO: 27 - protein, 7 aa, Gallus gallus
SRFSGRR

SEQ ID NO: 28 - protein, 9 aa, Gallus gallus
PGLALPVLP

SEQ ID NO: 29 - protein, 8 aa, Gallus gallus
SRVVIQPS

SEQ ID NO: 30 - protein, 8 aa, Gallus gallus
PILSSFPQ

SEQ ID NO: 31 - protein, 8 aa, Gallus gallus
LGSGIFWI

SEQ ID NO: 32 - protein, 9 aa, Gallus gallus
GISGLGSRF

SEQ ID NO: 33 - protein, 9 aa, Gallus gallus
VVVTLPGPI

SEQ ID NO: 34 - protein, 8 aa, Gallus gallus
CRLSLSQP

SEQ ID NO: 35 - protein, 11 aa, Gallus gallus
SAAVGSSLSAG

SEQ ID NO: 36 - protein, 11 aa, Gallus gallus
AVGSSTSAAVG

SEQ ID NO: 37 - protein, 9 aa, Gallus gallus
VVTLPGPIL

SEQ ID NO: 38 - protein, 9 aa, Gallus gallus
PGPILSSFP

SEQ ID NO: 39 - protein, 10 aa, Gallus gallus
SAAVGSILSE

SEQ ID NO: 40 - protein, 10 aa, Gallus gallus
VPISSGGFGI

SEQ ID NO: 41 - protein, 8 aa, Gallus gallus
QLAQRNTI

SEQ ID NO: 42 - protein, 9 aa, Gallus gallus
LSGLRSNLS

SEQ ID NO: 43 - protein, 10 aa, Gallus gallus
PSPVVVTLPG

SEQ ID NO: 44 - protein, 10 aa, Gallus gallus
PPPVVVTLPG

SEQ ID NO: 45 - protein, 10 aa, Gallus gallus
ISGLGSRFSG

SEQ ID NO: 46 - protein, 9 aa, Gallus gallus
LALRQSVEA

SEQ ID NO: 47 - protein, 10 aa, Gallus gallus
PLLGPPLLGI

SEQ ID NO: 48 - protein, 10 aa, Gallus gallus
VVTLPGPILS

SEQ ID NO: 49 - protein, 8 aa, Gallus gallus
VTLEDLRR

SEQ ID NO: 50 - protein, 9 aa, Gallus gallus
PILSSFPQN

SEQ ID NO: 51 - protein, 9 aa, Gallus gallus
PILSSFPQD

SEQ ID NO: 52 - protein, 11 aa, Gallus gallus
GTVGVSAGVTR

SEQ ID NO: 53 - protein, 9 aa, Gallus gallus
FGLSRVTLN

SEQ ID NO: 54 - protein, 12 aa, Gallus gallus
TAVGSSTSAAVG

SEQ ID NO: 55 - protein, 10 aa, Gallus gallus
VVVTLPGPIL

SEQ ID NO: 56 - protein, 10 aa, Gallus gallus
PPVVVTFPGP

SEQ ID NO: 57 - protein, 10 aa, Gallus gallus
PPPVVVTFPG

SEQ ID NO: 58 - protein, 10 aa, Gallus gallus
VVIQPSPVVV

SEQ ID NO: 59 - protein, 10 aa, Gallus gallus
FGISGLGSRF

SEQ ID NO: 60 - protein, 10 aa, Gallus gallus
PGPILSSFPQ

SEQ ID NO: 61 - protein, 10 aa, Gallus gallus
FGWSGLGRGL

SEQ ID NO: 62 - protein, 9 aa, Gallus gallus
LEAQLSELR

SEQ ID NO: 63 - protein, 9 aa, Gallus gallus
IVGAWYTVR

SEQ ID NO: 64 - protein, 11 aa, Gallus gallus
LLSPPGLPGLL

SEQ ID NO: 65 - protein, 11 aa, Gallus gallus
VVVTLPGPILS

SEQ ID NO: 66 - protein, 11 aa, Gallus gallus
GFGISGLGSRF

SEQ ID NO: 67 - protein, 11 aa, Gallus gallus
PVVVTLPGPIL

SEQ ID NO: 68 - protein, 11 aa, Gallus gallus
VSQGEITVPSI

SEQ ID NO: 69 - protein, 11 aa, Gallus gallus
SILSEEGVPIS

SEQ ID NO: 70 - protein, 11 aa, Gallus gallus
PVVVTFPGPIL

SEQ ID NO: 71 - protein, 13 aa, Gallus gallus
PISSGGFGISGLG

SEQ ID NO: 72 - protein, 13 aa, Gallus gallus
VGSSTSAAVGSIL

SEQ ID NO: 73 - protein, 11 aa, Gallus gallus
GFLSSAVSLNR

SEQ ID NO: 74 - protein, 12 aa, Gallus gallus
GGFGISGLGSRF

SEQ ID NO: 75 - protein, 11 aa, Gallus gallus
PGPILSSFPQN

SEQ ID NO: 76 - protein, 11 aa, Gallus gallus
PGPILSSFPQD

SEQ ID NO: 77 - protein, 12 aa, Gallus gallus
GGFGWSGLGRGL

SEQ ID NO: 78 - protein, 12 aa, Gallus gallus
PSPVVVTLPGPI

SEQ ID NO: 79 - protein, 12 aa, Gallus gallus
VVVTLPGPILSS

SEQ ID NO: 80 - protein, 12 aa, Gallus gallus
PVVVTLPGPILS

SEQ ID NO: 81 - protein, 12 aa, Gallus gallus
SPVVVTLPGPIL

SEQ ID NO: 82 - protein, 11 aa, Gallus gallus
RVVIQPSPVVV

SEQ ID NO: 83 - protein, 11 aa, Gallus gallus
QALTEVYAKAN

SEQ ID NO: 84 - protein, 10 aa, Gallus gallus
IETLTQFLGW

SEQ ID NO: 85 - protein, 13 aa, Gallus gallus
GFGSRSLVGLGGT

SEQ ID NO: 86 - protein, 14 aa, Gallus gallus
AVGSSTSAAVGSIL

SEQ ID NO: 87 - protein, 12 aa, Gallus gallus
PVVVTFPGPILS

SEQ ID NO: 88 - protein, 14 aa, Gallus gallus
VGSSTSAAVGSILS

SEQ ID NO: 89 - protein, 13 aa, Gallus gallus
GFGISGLGSRFSG

SEQ ID NO: 90 - protein, 13 aa, Gallus gallus
SGGFGISGLGSRF

SEQ ID NO: 91 - protein, 14 aa, Gallus gallus
VPISSGGFGISGLG

SEQ ID NO: 92 - protein, 12 aa, Gallus gallus
VVIQPSPVVVTL

SEQ ID NO: 93 - protein, 13 aa, Gallus gallus
SILSEEGVPISSG

SEQ ID NO: 94 - protein, 13 aa, Gallus gallus
VMGCKAAGASRII

SEQ ID NO: 95 - protein, 13 aa, Gallus gallus
SPVVVTLPGPILS

SEQ ID NO: 96 - protein, 12 aa, Gallus gallus
SRVVIQPSPVVV

SEQ ID NO: 97 - protein, 13 aa, Gallus gallus
PSPVVVTLPGPIL

SEQ ID NO: 98 - protein, 12 aa, Gallus gallus
RVVIQPSPVVVT

SEQ ID NO: 99 - protein, 13 aa, Gallus gallus
PPPVVVTLPGPIL

SEQ ID NO: 100 - protein, 15 aa, Gallus gallus
GVPISSGGFGISGLG

SEQ ID NO: 101 - protein, 13 aa, Gallus gallus
VIQPSPVVVTLPG

SEQ ID NO: 102 - protein, 15 aa, Gallus gallus
AVGSSTSAAVGSILS

SEQ ID NO: 103 - protein, 15 aa, Gallus gallus
TAVGSSTSAAVGSIL

SEQ ID NO: 104 - protein, 14 aa, Gallus gallus
SGGFGISGLGSRFS

SEQ ID NO: 105 - protein, 13 aa, Gallus gallus
VVVTLPGPILSSF

SEQ ID NO: 106 - protein, 13 aa, Gallus gallus
PPPVVVTFPGPIL

SEQ ID NO: 107 - protein, 14 aa, Gallus gallus
GLGGFGWSGLGRGL

SEQ ID NO: 108 - protein, 13 aa, Gallus gallus
VTLPGPILSSFPQ

SEQ ID NO: 109 - protein, 14 aa, Gallus gallus
AVGSILSEEGVPIS

SEQ ID NO: 110 - protein, 14 aa, Gallus gallus
SLGLGGFGWSGLGR

SEQ ID NO: 111 - protein, 14 aa, Gallus gallus
PSPVVVTLPGPILS

SEQ ID NO: 112 - protein, 13 aa, Gallus gallus
SRVVIQPSPVVVT

SEQ ID NO: 113 - protein, 15 aa, Gallus gallus
SGGFGISGLGSRFSG

SEQ ID NO: 114 - protein, 14 aa, Gallus gallus
PPPVVVTLPGPILS

SEQ ID NO: 115 - protein, 14 aa, Gallus gallus
VAIQPPPVVVTLPG

SEQ ID NO: 116 - protein, 15 aa, Gallus gallus
PISSGGFGISGLGSR

SEQ ID NO: 117 - protein, 13 aa, Gallus gallus
PIVDVAEALLHVK

SEQ ID NO: 118 - protein, 14 aa, Gallus gallus
VVIQPSPVVVTLPG

SEQ ID NO: 119 - protein, 13 aa, Gallus gallus
RVVIQPSPVVVTL

SEQ ID NO: 120 - protein, 16 aa, Gallus gallus
TAVGSSTSAAVGSILS

SEQ ID NO: 121 - protein, 14 aa, Gallus gallus
PPPVVVTFPGPILS

SEQ ID NO: 122 - protein, 15 aa, Gallus gallus
SLGLGGFGWSGLGRG

SEQ ID NO: 123 - protein, 14 aa, Gallus gallus
VVVTLPGPILSSFP

SEQ ID NO: 124 - protein, 14 aa, Gallus gallus
PVVVTLPGPILSSF

SEQ ID NO: 125 - protein, 15 aa, Gallus gallus
AAVGSILSEEGVPIS

SEQ ID NO: 126 - protein, 15 aa, Gallus gallus
SAAVGSILSEEGVPI

SEQ ID NO: 127 - protein, 16 aa, Gallus gallus
EGVPISSGGFGISGLG

SEQ ID NO: 128 - protein, 14 aa, Gallus gallus
VVTLPGPILSSFPQ

SEQ ID NO: 129 - protein, 15 aa, Gallus gallus
PSPVVVTLPGPILSS

SEQ ID NO: 130 - protein, 16 aa, Gallus gallus
GKDGPKGVRGDAGPPG

SEQ ID NO: 131 - protein, 15 aa, Gallus gallus
SVAIQPPPVVVTLPG

SEQ ID NO: 132 - protein, 14 aa, Gallus gallus
SRVVIQPSPVVVTL

SEQ ID NO: 133 - protein, 14 aa, Gallus gallus
LHTEAAATVPDRSE

SEQ ID NO: 134 - protein, 15 aa, Gallus gallus
VVIQPSPVVVTLPGP

SEQ ID NO: 135 - protein, 14 aa, Gallus gallus
RVVIQPSPVVVTLP

SEQ ID NO: 136 - protein, 16 aa, Gallus gallus
SAAVGSILSEEGVPIS

SEQ ID NO: 137 - protein, 15 aa, Gallus gallus
PVVVTLPGPILSSFP

SEQ ID NO: 138 - protein, 15 aa, Gallus gallus
IQPSPVVVTLPGPIL

SEQ ID NO: 139 - protein, 17 aa, Gallus gallus
SVPIGAGGSLGLGGFGW

SEQ ID NO: 140 - protein, 16 aa, Gallus gallus
SLGLGGFGWSGLGRGL

SEQ ID NO: 141 - protein, 17 aa, Gallus gallus
FGGGFGGGDGILPAGEK

SEQ ID NO: 142 - protein, 17 aa, Gallus gallus
TAVGSSTSAAVGSILSE

SEQ ID NO: 143 - protein, 16 aa, Gallus gallus
PISSGGFGISGLGSRF

SEQ ID NO: 144 - protein, 19 aa, Gallus gallus
SAAVGSSLSAGSVPIGAGG

SEQ ID NO: 145 - protein, 17 aa, Gallus gallus
GVPISSGGFGISGLGSR

SEQ ID NO: 146 - protein, 15 aa, Gallus gallus
VVVTLPGPILSSFPQ

SEQ ID NO: 147 - protein, 15 aa, Gallus gallus
RVVIQPSPVVVTLPG

SEQ ID NO: 148 - protein, 15 aa, Gallus gallus
SRVVIQPSPVVVTLP

SEQ ID NO: 149 - protein, 16 aa, Gallus gallus
PSPVVVTLPGPILSSF

SEQ ID NO: 150 - protein, 16 aa, Gallus gallus
AIQPPPVVVTLPGPIL

SEQ ID NO: 151 - protein, 16 aa, Gallus gallus
IQPSPVVVTLPGPILS

SEQ ID NO: 152 - protein, 16 aa, Gallus gallus
VIQPSPVVVTLPGPIL

SEQ ID NO: 153 - protein, 17 aa, Gallus gallus
VPISSGGFGISGLGSRF

SEQ ID NO: 154 - protein, 16 aa, Gallus gallus
SRVVIQPSPVVVTLPG

SEQ ID NO: 155 - protein, 17 aa, Gallus gallus
SILSEEGVPISSGGFGI

SEQ ID NO: 156 - protein, 16 aa, Gallus gallus
PVVVTLPGPILSSFPQ

SEQ ID NO: 157 - protein, 16 aa, Gallus gallus
RVVIQPSPVVVTLPGP

SEQ ID NO: 158 - protein, 18 aa, Gallus gallus
SAAVGSILSEEGVPISSG

SEQ ID NO: 159 - protein, 16 aa, Gallus gallus
VVVTLPGPILSSFPQN

SEQ ID NO: 160 - protein, 18 aa, Gallus gallus
GVPISSGGFGISGLGSRF

SEQ ID NO: 161 - protein, 17 aa, Gallus gallus
PSPVVVTLPGPILSSFP

SEQ ID NO: 162 - protein, 17 aa, Gallus gallus
VAIQPPPVVVTLPGPIL

SEQ ID NO: 163 - protein, 17 aa, Gallus gallus
VIQPSPVVVTLPGPILS

SEQ ID NO: 164 - protein, 17 aa, Gallus gallus
VVIQPSPVVVTLPGPIL

SEQ ID NO: 165 - protein, 17 aa, Gallus gallus
SPVVVTLPGPILSSFPQ

SEQ ID NO: 166 - protein, 15 aa, Gallus gallus
VILSNTKTQYSIKIH

SEQ ID NO: 167 - protein, 17 aa, Gallus gallus
SRVVIQPSPVVVTLPGP

SEQ ID NO: 168 - protein, 17 aa, Gallus gallus
DSRVVIQPSPVVVTLPG

SEQ ID NO: 169 - protein, 17 aa, Gallus gallus
PVVVTLPGPILSSFPQN

SEQ ID NO: 170 - protein, 17 aa, Gallus gallus
RVVIQPSPVVVTLPGPI

SEQ ID NO: 171 - protein, 19 aa, Gallus gallus
GVPISSGGFGISGLGSRFS

SEQ ID NO: 172 - protein, 18 aa, Gallus gallus
SVAIQPPPVVVTLPGPIL

SEQ ID NO: 173 - protein, 18 aa, Gallus gallus
VAIQPPPVVVTLPGPILS

SEQ ID NO: 174 - protein, 18 aa, Gallus gallus
VVIQPSPVVVTLPGPILS

SEQ ID NO: 175 - protein, 18 aa, Gallus gallus
PSPVVVTLPGPILSSFPQ

SEQ ID NO: 176 - protein, 20 aa, Gallus gallus
GVPISSGGFGISGLGSRFSG

SEQ ID NO: 177 - protein, 18 aa, Gallus gallus
AVPLVRPVTMVPSIPGIP

SEQ ID NO: 178 - protein, 18 aa, Gallus gallus
PPPVVVTLPGPILSSFPQ

SEQ ID NO: 179 - protein, 18 aa, Gallus gallus
SPVVVTLPGPILSSFPQN

SEQ ID NO: 180 - protein, 18 aa, Gallus gallus
LPGPGLQLLHGTTTLAFK

SEQ ID NO: 181 - protein, 18 aa, Gallus gallus
PPPVVVTFPGPILSSFPQ

SEQ ID NO: 182 - protein, 19 aa, Gallus gallus
SVAIQPPPVVVTLPGPILS

SEQ ID NO: 183 - protein, 19 aa, Gallus gallus
SSVAIQPPPVVVTLPGPIL

SEQ ID NO: 184 - protein, 18 aa, Gallus gallus
RVVIQPSPVVVTLPGPIL

SEQ ID NO: 185 - protein, 19 aa, Gallus gallus
PSPVVVTLPGPILSSFPQN

SEQ ID NO: 186 - protein, 21 aa, Gallus gallus
SILSEEGVPISSGGFGISGLG

SEQ ID NO: 187 - protein, 19 aa, Gallus gallus
SRVVIQPSPVVVTLPGPIL

SEQ ID NO: 188 - protein, 19 aa, Gallus gallus
RVVIQPSPVVVTLPGPILS

SEQ ID NO: 189 - protein, 22 aa, Gallus gallus
SVPIGAGGSLGLGGFGWSGLGR

SEQ ID NO: 190 - protein, 22 aa, Gallus gallus
TAVGSSTSAAVGSILSEEGVPI

SEQ ID NO: 191 - protein, 22 aa, Gallus gallus
SAAVGSILSEEGVPISSGGFGI

SEQ ID NO: 192 - protein, 21 aa, Gallus gallus
SEEGVPISSGGFGISGLGSRF

SEQ ID NO: 193 - protein, 20 aa, Gallus gallus
SRVVIQPSPVVVTLPGPILS

SEQ ID NO: 194 - protein, 23 aa, Gallus gallus
SVPIGAGGSLGLGGFGWSGLGRG

SEQ ID NO: 195 - protein, 20 aa, Gallus gallus
DSRVVIQPSPVVVTLPGPIL

SEQ ID NO: 196 - protein, 23 aa, Gallus gallus
TAVGSSTSAAVGSILSEEGVPIS

SEQ ID NO: 197 - protein, 22 aa, Gallus gallus
LRGDVGPVGRTGEQGIAGPPGF

SEQ ID NO: 198 - protein, 21 aa, Gallus gallus
SRVVIQPSPVVVTLPGPILSS

SEQ ID NO: 199 - protein, 21 aa, Gallus gallus
VVIQPSPVVVTLPGPILSSFP

SEQ ID NO: 200 - protein, 22 aa, Gallus gallus
GVPISSGGFGISGLGSRFSGRR

SEQ ID NO: 201 - protein, 24 aa, Gallus gallus
SVPIGAGGSLGLGGFGWSGLGRGL

SEQ ID NO: 202 - protein, 21 aa, Gallus gallus
DSRVVIQPSPVVVTLPGPILS

SEQ ID NO: 203 - protein, 21 aa, Gallus gallus
VIQPSPVVVTLPGPILSSFPQ

SEQ ID NO: 204 - protein, 24 aa, Gallus gallus
AVGSILSEEGVPISSGGFGISGLG

SEQ ID NO: 205 - protein, 21 aa, Gallus gallus
RVVIQPSPVVVTLPGPILSSF

SEQ ID NO: 206 - protein, 23 aa, Gallus gallus
SILSEEGVPISSGGFGISGLGSR

SEQ ID NO: 207 - protein, 21 aa, Gallus gallus
IPSSVPESSFKIKGNSSSFHI

SEQ ID NO: 208 - protein, 22 aa, Gallus gallus
VAIQPPPVVVTLPGPILSSFPQ

SEQ ID NO: 209 - protein, 25 aa, Gallus gallus
TAVGSSTSAAVGSILSEEGVPISSG

SEQ ID NO: 210 - protein, 22 aa, Gallus gallus
VVIQPSPVVVTLPGPILSSFPQ

SEQ ID NO: 211 - protein, 23 aa, Gallus gallus
PSPVVVTLPGPILSSFPQNTAVG

SEQ ID NO: 212 - protein, 20 aa, Gallus gallus
KENFEVLCKDGTRKPVTDAE

SEQ ID NO: 213 - protein, 21 aa, Gallus gallus
SIQACKLAQSHGWGVMVSHRS

SEQ ID NO: 214 - protein, 22 aa, Gallus gallus
SRVVIQPSPVVVTLPGPILSSF

SEQ ID NO: 215 - protein, 22 aa, Gallus gallus
RVVIQPSPVVVTLPGPILSSFP

SEQ ID NO: 216 - protein, 23 aa, Gallus gallus
EQPPLCVASVSVKEPASETPAVP

SEQ ID NO: 217 - protein, 23 aa, Gallus gallus
SVAIQPPPVVVTLPGPILSSFPQ

SEQ ID NO: 218 - protein, 26 aa, Gallus gallus
SAAVGSILSEEGVPISSGGFGISGLG

SEQ ID NO: 219 - protein, 24 aa, Gallus gallus
SILSEEGVPISSGGFGISGLGSRF

SEQ ID NO: 220 - protein, 24 aa, Gallus gallus
PSPVVVTLPGPILSSFPQNTAVGS

SEQ ID NO: 221 - protein, 23 aa, Gallus gallus
VVIQPSPVVVTLPGPILSSFPQN

SEQ ID NO: 222 - protein, 23 aa, Gallus gallus
SRVVIQPSPVVVTLPGPILSSFP

SEQ ID NO: 223 - protein, 24 aa, Gallus gallus
SSVAIQPPPVVVTLPGPILSSFPQ

SEQ ID NO: 224 - protein, 23 aa, Gallus gallus
RVVIQPSPVVVTLPGPILSSFPQ

SEQ ID NO: 225 - protein, 25 aa, Gallus gallus
SILSEEGVPISSGGFGISGLGSRFS

SEQ ID NO: 226 - protein, 27 aa, Gallus gallus
TSAAVGSILSEEGVPISSGGFGISGLG

SEQ ID NO: 227 - protein, 26 aa, Gallus gallus
SILSEEGVPISSGGFGISGLGSRFSG

SEQ ID NO: 228 - protein, 24 aa, Gallus gallus
SRVVIQPSPVVVTLPGPILSSFPQ

SEQ ID NO: 229 - protein, 28 aa, Gallus gallus
TAVGSSTSAAVGSILSEEGVPISSGGFG

SEQ ID NO: 230 - protein, 28 aa, Gallus gallus
STSAAVGSILSEEGVPISSGGFGISGLG

SEQ ID NO: 231 - protein, 24 aa, Gallus gallus
RVVIQPSPVVVTLPGPILSSFPQN

SEQ ID NO: 232 - protein, 25 aa, Gallus gallus
SRVVIQPSPVVVTLPGPILSSFPQN

SEQ ID NO: 233 - protein, 25 aa, Gallus gallus
DSRVVIQPSPVVVTLPGPILSSFPQ

SEQ ID NO: 234 - protein, 29 aa, Gallus gallus
TAVGSSTSAAVGSILSEEGVPISSGGFGI

SEQ ID NO: 235 - protein, 29 aa, Gallus gallus
SAAVGSILSEEGVPISSGGFGISGLGSRF

SEQ ID NO: 236 - protein, 24 aa, Gallus gallus
VEQLVNLQHLDVAYNLLLEHAQLA

SEQ ID NO: 237 - protein, 28 aa, Gallus gallus
SVAIQPPPVVVTLPGPILSSFPQSTAVG

SEQ ID NO: 238 - protein, 28 aa, Gallus gallus
SILSEEGVPISSGGFGISGLGSRFSGRR

SEQ ID NO: 239 - protein, 30 aa, Gallus gallus
TSAAVGSILSEEGVPISSGGFGISGLGSRF

SEQ ID NO: 240 - protein, 29 aa, Gallus gallus
SSVAIQPPPVVVTLPGPILSSFPQSTAVG

SEQ ID NO: 241 - protein, 31 aa, Gallus gallus
STSAAVGSILSEEGVPISSGGFGISGLGSRF

SEQ ID NO: 242 - protein, 31 aa, Gallus gallus
PSPVVVTLPGPILSSFPQNTAVGSSTSAAVG

SEQ ID NO: 243 - protein, 33 aa, Gallus gallus
TAVGSSTSAAVGSILSEEGVPISSGGFGISGLG

SEQ ID NO: 244 - protein, 31 aa, Gallus gallus
AVGSILSEEGVPISSGGFGISGLGSRFSGRR

SEQ ID NO: 245 - protein, 27 aa, Gallus gallus
NKVLVHCAMGRSRSATLVLAYLMIYKN

SEQ ID NO: 246 - protein, 32 aa, Gallus gallus
SSVAIQPPPVVVTLPGPILSSFPQSTAVGSTA

SEQ ID NO: 247 - protein, 32 aa, Gallus gallus
AAVGSILSEEGVPISSGGFGISGLGSRFSGRR

SEQ ID NO: 248 - protein, 35 aa, Gallus gallus
TAVGSSTSAAVGSILSEEGVPISSGGFGISGLGSR

SEQ ID NO: 249 - protein, 33 aa, Gallus gallus
SAAVGSILSEEGVPISSGGFGISGLGSRFSGRR

SEQ ID NO: 250 - protein, 34 aa, Gallus gallus
PSPVVVTLPGPILSSFPQNTAVGSSTSAAVGSIL

SEQ ID NO: 251 - protein, 35 aa, Gallus gallus
PSPVVVTLPGPILSSFPQNTAVGSSTSAAVGSILS

SEQ ID NO: 252 - protein, 36 aa, Gallus gallus
TAVGSSTSAAVGSILSEEGVPISSGGFGISGLGSRF

SEQ ID NO: 253 - protein, 29 aa, Gallus gallus
RPRVYLHCTNPRHPKYREGDVTLFALNLS